(19) United States
(12) Patent Application Publication
(10) Pub. No.: US 2011/0269727 A1
(43) Pub. Date: Nov. 3, 2011

(54) COMPOSITION TO REDUCE ALLODYNIC BACK PAIN AND RELATED METHOD OF USE

(76) Inventor: Annette C. Toledano, North Miami, FL (US)

(21) Appl. No.: 12/824,367

(22) Filed: Jun. 28, 2010

Related U.S. Application Data
(60) Provisional application No. 61/343,489, filed on Apr. 29, 2010.
(60) Provisional application No. 61/395,772, filed on May 17, 2010.

(51) Int. Cl. A61K 31/56; A61K 31/498; A61K 31/439; A61K 31/451; A61P 21/02; A61P 25/24; A61P 25/08; A61P 11/10; A61P 11/02; A61P 29/00; A61K 31/54; A61P 11/14

(52) U.S. Cl. 514/171; 514/227.2; 514/249; 514/282; 514/331

(57) ABSTRACT

The invention relates to a pharmaceutical composition to reduce back pain comprising two compounds: an opioid antagonist and a direct-acting alpha 2 adrenegic agonist. The Opioid antagonist is selected from the group consisting of alvimopan, nalmefene, naloxone, naltrexone, methylnaltrexone, nalorphine, and pharmaceutically acceptable salt. The direct-acting alpha 2 adrenegic agonist is selected from a group consisting of Apraclonidine, Brimonidine, Clonidine, Detomidine, Dexmedetomidine, Guanabenz, Guanfacine, Lofexidine, Medetomidine, Romifidine, Tizanidine, Tolonidine, Xylazine and Fadolmidine. In one embodiment, the composition may include naltrexone (or its pharmaceutically acceptable salt) as the opioid antagonist and clonidine hydrochloride (or its pharmaceutically acceptable salt) as the direct-acting alpha 2 adrenegic agonist. It is preferred naltrexone be administered with a second agent such as an antitussive, expectorant, decongestant, or antihistamine. The dosage of naltrexone may range between 0.25 mg to 15 mg, while the dosage of clonidine hydrochloride ranges between 0.0125 mg and 0.3 mg.

### CROSS-REFERENCE TO RELATED APPLICATION

The application seeks priority to U.S. Provisional Application Ser. No. 61/343,489 entitled “Methods For Treating Pain By Composition of Naltrexone/Clonidine At Any Dose Combination” filed on Apr. 29, 2010 and U.S. Provisional Application Ser. No. 61/395,772 entitled “Pharmaceutical Combinations for Alleviation of Back Pain also called Spinal Pain” filed on May 17, 2010, the contents of which are hereby both incorporated by reference in their entirety.

This invention is directed to a pharmaceutical composition of two compounds to alleviate back pain. More specifically, the composition teaches combination of a low dose Opioid antagonist, Naltrexone, and an alpha two adrenergic receptor agonist, Clonidine, to reduce back pain.

### BACKGROUND OF THE INVENTION

Back pain represents one of the most common and chronic physical ailments. In the United States, acute back pain (also referred to as “lumbago”) is the fifth most common reason for physician visits. In fact, nine of ten adults experience back pain at some point in their life, and five out of ten working adults report back pain every year.

Observational studies suggest two conditions to which back pain has been attributed. The first is lumbar disc herniation and degenerative disc disease, where the underlying mechanisms causing the pain remain unknown. Studies suggest that for as many as 85 percent of cases, no physiological cause can be shown. There are several potential sources and causes of back pain. However, the diagnosis of specific tissues of the spine as the cause of pain presents problems. This is because symptoms arising from different spinal tissues can feel very similar and is difficult to differentiate without the use of invasive diagnostic intervention procedures, such as local anesthetic blocks.

One potential source of back pain is attributed to skeletal muscle in the back. Causes this type of back pain include muscle strains, spasm, and imbalances. However, imaging studies do not support the notion of muscle tissue damage in many back pain cases, and the neurophysiology of muscle spasm and imbalances are not well understood.

Another potential source of low back pain is the synovial joints of the spine (e.g., zygapophysial joints). These have been identified as the primary source of the pain in approximately one third of people with chronic low back pain, and in most people with neck pain following whiplash. However, the cause of zygapophysial joint pain is not fully understood. Capsule tissue damage has been proposed in people with neck pain following whiplash. In people with spinal pain stemming from zygapophysial joints, one theory is that intra-articular tissue such as invaginations of their synovial membranes and fibro-adipose meniscoids (that usually act as a cushion to help the bones move over each other smoothly) may become displaced, pinched or trapped, and consequently give rise to nociception.

New attention has been focused on non-discogenic back pain, where patients have normal or near-normal MRI and CT scans. One of the newer investigations looks into the role of the dorsal ramus in patients that have no radiographic abnormalities. The management goals when treating back pain are to achieve maximal reduction in pain intensity as rapidly as possible; to restore the individual's ability to function in everyday activities; to help the patient cope with residual pain; to assess for side-effects of therapy; and to facilitate the patient's passage through the legal and socioeconomic impediments to recovery. For many, the goal is to keep the pain to a manageable level to progress with rehabilitation, which then can lead to long term pain relief. Also, for some people the goal is to use non-surgical therapies to manage the pain and avoid major surgery, while for others surgery may be the quickest way to feel better.

While only a minority of individuals suffering from back pain resort to surgery, there are only limited options for offering relief from chronic back pain. Heat therapy is one option for short term relief, as well as alternative cold compression therapy proximate to the location of pain. Alternative treatments such as message therapy, acupressure and pressure point message have also been used. Some pharmaceuticals have shown usefulness in treating back pain, including muscle relaxants, non-steroidal anti-inflammatory drugs, analgesics and Opioid agonists.

Accordingly, there is a need in the art of treating back pain for an alternative pharmaceutical composition that reduces the symptoms of back pain effectively for longer periods of time and is not a narcotic compound.

### SUMMARY OF THE INVENTION

The present invention offers an alternative pharmaceutical composition which reduces the symptoms of back pain. The composition includes two compounds: an Opioid antagonist and a direct-acting alpha 2 adrenegic agonist. The Opioid antagonist is selected from the group consisting of alvimopan, nalmefene, naloxone, naltrexone, methylnaltrexone, nalorphine, and pharmaceutically acceptable salt. The direct-acting alpha 2 adrenegic agonist is selected from a group consisting of Apraclonidine, Brimonidine, Clonidine, Detomidine, Dexmedetomidine, Guanabenz, Guanfacine, Lofexidine, Medetomidine, Romifidine, Tizanidine, Tolonidine, Xylazine and Fadolmidine.

In one embodiment, the composition may include naltrexone (or its pharmaceutically acceptable salt) as the opioid antagonist, and clonidine hydrochloride (or its pharmaceutically acceptable salt) as the direct-acting alpha 2 adrenegic agonist. It is preferred that naltrexone be administered in low doses, along with a second pharmaceutical agent such as an non steroidal anti inflammatory drug (NSAID) of all chemical groups, including COX-2 selective inhibitors(coxibs), steroidal anti inflammatory drugs, Tricyclic antidepressants (TCAs), Selective serotonin reuptake inhibitors (SSRIs), Serotonin-norepinephrine reuptake inhibitors (SNRIs), Anticonvulsants, muscle relaxant, drug with NMDA antagonist properties, Tetrahydrocannabinol derivatives, antitussive, an expectorant, a decongestant, or an antihistamine. The dosage of naltrexone may range between 0.25 mg to 15 mg, while the dosage of clonidine hydrochloride ranges between 0.0125 and 0.3 mg.

The invention is further directed to a method for reducing back pain. The first step includes administering a first compound which includes an opioid antagonist. The second step includes taking a second compound which includes a direct-acting alpha 2 adrenegic agonist. As described above, the opioid antagonist can be a low dose of naltrexone in the range of 0.25 mg to 15 mg, while the direct-acting alpha 2 adrenegic agonist is a dose of clonidine hydrochloride ranging between 0.0125 and 0.3 mg. The method further contemplates administering a dosage of both compositions once during the daytime, with a second administration proximate to bedtime to maximize the results.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

The Pharmaceutical Composition

This invention is directed to a pharmaceutical composition of two compounds which help alleviate and reduce the systems commonly associated with back pain. The specific focus of the present invention is the treatment of back pain, which is a type of alloynia. Allodynia means “other pain,” and is defined as pain due to a stimulus which does not normally provoke pain.

Different cell types have been linked to allodynia. Recent reports that microglia in the thalamus might contribute to allodynia by changing the properties of the secondary nociceptors. The same effect is achieved in the spinal cord by the recruitment of immune system cells such as monocytes/macrophages and T lymphocytes. There is a strong body of evidence that the so called sensitization of the central nervous system contributes to the appearance of allodynia. Sensitization refers to the increased response of neurons following repetitive stimulation.

In addition to repeated activity, the increased levels of certain compounds lead to sensitization, as well. The work of many researchers has led to the elucidation of pathways that can result in neuronal sensitization both in the thalamus and dorsal horns. Both pathways depend on the production of chemokines and other molecules important in the inflammatory response.

A very important molecule in the thalamus appears to be cysteine-cysteine chemokine ligand 21 (CCL21). The concentration of this chemokine is increased in the ventral posterolateral nucleus of the thalamus where secondary nociceptive neurons make connections with other neurons. The source of CCL21 is not exactly known, but two possibilities exist. First, it might be made in primary nociceptive neurons and transported up to the thalamus. Most likely, neurons intrinsic to the ventral posterolateral nucleus make at least some of it. In any case, CCL21 binds to C-C chemokine receptor type 7 and chemokine receptor CXCR3 receptors on microglia in the thalamus. The physiologic response to the binding is probably the production of prostaglandin E2 (PGE2) by cyclooxygenase 2 (COX-2). Activated microglia making PGE2 can then sensitize nociceptive neurons as manifested by their lowered threshold to pain.

The mechanism responsible for sensitization of the central nervous system at the level of the spinal cord is different from the one in the thalamus. Tumor necrosis factor-alpha (TNF-alpha) and its receptor are the molecules that seem to be responsible for the sensitization of neurons in the dorsal horns of the spinal cord. Macrophages and lymphocytes infiltrate the spinal cord, for example, because of injury, and release TNF-alpha and other pro-inflammatory molecules [15] TNF-alpha then binds to the TNF receptors expressed on nociceptors, activating the MAPK/NF-kappa B pathways. This leads to the production of more TNF-alpha, its release, and binding to the receptors on the cells that released it (autocrine signaling).

This mechanism also explains the perpetuation of sensitization and thus allodynia. TNF-alpha might also increase the number of AMPA receptors, and decrease the numbers of GABA receptors on the membrane of nociceptors, both of which could change the nociceptors in a way that allows for their easier activation. Another outcome of the increased TNF-alpha is the release of PGE2, with a mechanism and effect similar to the ones in the thalamus.

Based upon this, the invention first teaches use of an opioid antagonist in order to sensitize nociceptive neurons to in turn lower the sensation of back pain. Specifically, the invention contemplates several forms of opioid antagonist selected from a group consisting of alvimopan, nalmefene, naloxone, naltrexone, methylnaltrexone, nalorphine, and pharmaceutically acceptable salt.

Low dose naltrexone and its pharmaceutically acceptable salt are contemplated as the primary choice for the opioid antagonist. Currently, low dose naltrexone is used in drug/alcohol rehabilitation purposes, as well as treating certain immunologically-related disorders, including HIV/AIDS and multiple sclerosis. Naltrexone has also been shown to have positive results in the treatment of fibromyalgia, autoimmune diseases such as rheumatoid arthritis, ankylosing spondylitis, Crohn's disease, ulcerative colitis, Hashimoto's thyroiditis, and certain central nervous system disorders.

Here, the invention contemplates use of naltrexone hydrochloride in crystalline or amorphous form with a dosage ranging between 0.25 mg to 15 mg. Such dosage can be combined with a second pharmaceutical agent selected from a group consisting of an non steroidal anti inflammatory drug (NSAID) of all chemical groups, including COX-2 selective inhibitor(coxibs), steroidal anti inflammatory drugs, Tricyclic antidepressants (TCAs), Selective serotonin reuptake inhibitors (SSRIs), Serotonin-norepinephrine reuptake inhibitors (SNRIs), Anticonvulsants, muscle relaxant, drug with NMDA antagonist properties, Tetrahydrocannabinol derivatives, antitussive, an expectorant, a decongestant, or an antihistamine.

Apart from opioid antagonist, the invention contemplates administration of a direct-acting alpha 2 adrenegic agonist as a second compound to relief back pain. The direct-acting alpha 2 adrenegic agonist is selected from a group consisting of Apraclonidine, Brimonidine, Clonidine, Detomidine, Dexmedetomidine, Guanabenz, Guanfacine, Lofexidine, Medetomidine, Romifidine, Tizanidine, Tolonidine, Xylazine and Fadolmidine. The invention specifically contemplates clonidine hydrochloride or its pharmaceutically acceptable salt as the direct-acting alpha 2 adrenegic agonist. More specifically, the dosage of clonidine hydrochloride ranges contemplated between 0.0125 and 0.3 mg.

Clonidine is a drug commonly used to treat high blood pressure—has been shown to effectively treat neuropathic pain, is FDA-approved for administration via epidural (an injection given in the lower back), and is the third most commonly prescribed drug for chronic intrathecal (an injection into the cerebrospinal fluid) use in people with chronic pain.

Accordingly, the invention teaches a pharmaceutical compound to reduce back pain with a single unit dosage of a low dose quantity of naltrexone (or its pharmaceutically acceptable salt) and a quantity of clonidine hydrochloride (or its pharmaceutically acceptable salt). The dosage of these two primary pharmaceutical compounds can be orally through pill, through a liquid or transdermal gel, or through injection via syringe. For more immediate results, treatment can be performed by a professional through epidural.

Method of Use

The invention is further directed to a method for reducing back pain. The first step of the method is to administer a first compound which includes an opoid antagonist selected from the group consisting of alvimopan, nalmefene, naloxone, naltrexone, methylnaltrexone, nalorphine, and pharmaceutically acceptable salt. The second step is to take a second compound which includes a direct-acting alpha 2 adrenegic agonist selected from the group consisting of Apraclonidine, Brimonidine, Clonidine, Detomidine, Dexmedetomidine, Guanabenz, Guanfacine, Lofexidine, Medetomidine, Romifidine, Tizanidine, Tolonidine, Xylazine and Fadolmidine. Specifically, the invention contemplates administering a low dose of naltrexone in the range of 0.25 mg to 15 mg, and then later taking a dose of clonidine hydrochloride ranging between 0.0125 and 0.3 mg.

The method can further include a regiment of taking a dosage of both compounds in the morning/daytime, with a second administration proximate to bedtime. The ratio of naltrezone to clonidine dosage contemplated for the initial morning/daytime administration of the pharmaceutical ranges as illustrated by reference to Table 1:

TABLE 1

| Clonidine Hydrochloride | Naltrexone Hydrochloride |
|---|---|
| 0.0125 mg | 1 mg |
| 0.025 mg | 1 mg |
| 0.025 mg | 2 mg |
| 0.025 mg | 3 mg |
| 0.05 mg | 3 mg |

Similarly, the ratio of naltrexone to clonidine dosage contemplated for the secondary nighttime administration of the pharmaceutical ranges illustrated by reference to Table 2:

TABLE 2

| Clonidine Hydrochloride | Naltrexone Hydrochloride |
|---|---|
| 0.05 | 1 mg |
| 0.1 | 1 mg |
| 0.2 | 1 mg |

Pilot Study

In a pilot study fifteen patients with a diagnosis of back pain of cervical and/or lumbar areas coexisting with /without headache or osteoarthritic joint pain (hip or knee) were studied. A control group of 15 patients received physical therapy. The test had patients rank their back pain based upon a “0-10 numeric pain intensity scale” where 10 represented intolerable pain.

The study group received a dose of the composition and the control group received physical therapy with following modalities: hot pack, electrical muscle stimulation, ultrasound, manual electrical muscle stimulation and massage to an affected areas for 30 minutes to 45 minutes. The “0-10 numeric pain intensity scale” was administered before and 30-60 minutes after administration of composition orally. The control group responded to the “0-10 numeric pain intensity scale” before and immediately after physical therapy to one area.

The results of this pilot study showing the improved mood and reduced headaches of patients suffering from back pain are illustrated by reference to Table 3:

TABLE 3

| | Cervical/ | Lumbar/ | Joint/ | Mood/ | |
|---|---|---|---|---|---|
| Average | number of | number | number of | number of | |
| improvement | patients | of patients | patients | patients | Headache |
| Study | −4.75/12 | −4.33/12 | −6/4 | 4.72/11 | −5/4 |
| group. (15) | | | | | |
| Control | −1.2/10 | −0.81/11 | −0/9 | 0.4/5 | 0/4 |
| group, | | | | | |
| physical | | | | | |
| therapy. (15) | | | | | |

The group that received the composition had a reduction of 4.75 points of the “0-10 Numerical Pain Intensity Scale” in the cervical area, 4.3 point reduction in the lumbar area. 6 point reduction in joint (such as knee or hip joint)and 5 point reduction in headache. There was 4.72 improvement in mood (10—good mood). The group that received physical therapy had a reduction of 0.81-1.2 points of “0-10 Numerical Pain Intensity Scale”. This finding has a P value of <0.000001, extremely statistically significant difference between group treated with composition and the group treated with physical therapy.

The composition treated group has done better than just being compared to placebo, it was compared to a treatment that is considered to be helpful. Although the peak benefit of the composition is not until 3-4 hours after administration the results were obtained only 30-60 minutes after administration and there for under estimate the full effect of the composition on back pain. The effect of physical therapy is maximal immediately after treatment. There was 4.7 point improvement in mood comparing to 0.4 points in control. Accordingly, the results are striking and reveal a high efficacy of this invention.

1. A pharmaceutical formulation to reduce back pain, the formulation comprising:
a first compound which includes an opioid antagonist; and
a second compound which includes a direct-acting alpha 2 adrenegic agonist.

2. The formulation of claim 1, wherein the opioid antagonist is selected from a group consisting of alvimopan, nalmefene, naloxone, naltrexone, methylnaltrexone, nalorphine, and pharmaceutically acceptable salt.

3. The formulation of claim 1, wherein the opioid antagonist is naltrexone or its pharmaceutically acceptable salt.

4. The formulation of claim 1, wherein the opioid antagonist is naltrexone hydrochloride in crystalline or amorphous form.

5. The formulation of claim 1, wherein the direct-acting alpha 2 adrenegic agonist is selected from a group consisting of Apraclonidine, Brimonidine, Clonidine, Detomidine, Dexmedetomidine, Guanabenz, Guanfacine, Lofexidine, Medetomidine, Romifidine, Tizanidine, Tolonidine, Xylazine and Fadolmidine.

6. The formulation of claim 1, wherein the direct-acting alpha 2 adrenegic agonist is clonidine or its pharmaceutically acceptable salt.

7. The formulation of claim 1, wherein the direct-acting alpha 2 adrenegic agonist is clonidine hydrochloride or its pharmaceutically acceptable salt.

8. The formulation of claim 1, wherein the opioid antagonist is a quantity of naltrexone in combination with second pharmaceutical agent selected from a group consisting of a non steroidal anti inflammatory drug (NSAID) of all chemical groups, including COX-2 selective inhibitor(coxibs),steroidal anti inflammatory drugs, Tricyclic antidepressants (TCAs), Selective serotonin reuptake inhibitors (SSRIs), Serotonin-norepinephrine reuptake inhibitors (SNRIs), Anticonvulsants, muscle relaxant, drug with NMDA antagonist properties, Tetrahydrocannabinol derivatives, an antitussive, an expectorant, a decongestant, or an antihistamine.

9. A formulation compound to reduce back pain, comprising a single unit dosage of:
a quantity of naltrexone or its pharmaceutically acceptable salt; and
a quantity of clonidine hydrochloride or its pharmaceutically acceptable salt.

10. The formulation of claim 9, wherein the dosage of naltrexone ranges between 0.25 mg to 15 mg.

11. The formulation of claim 9, wherein the dosage of naltrexone hydrochloride ranges between 0.25 mg to 15 mg.

12. he formulation of claim 9, wherein the dosage of clonidine hydrochloride ranges between 0.0125 and 0.3 mg.

13. A method for reducing back pain, comprising the steps of:
(a) administering a first compound which includes an opioid antagonist; and
(b) taking a second compound which includes a direct-acting alpha 2 adrenegic agonist.

14. The method of claim 13, wherein the opioid antagonist is selected from a group consisting of alvimopan, nalmefene, naloxone, naltrexone, methylnaltrexone, nalorphine, and pharmaceutically acceptable salt.

15. he method of claim 13, wherein the opioid antagonist is naltrexone or its pharmaceutically acceptable salt.

16. he method of claim 13, wherein the opioid antagonist is naltrexone hydrochloride in crystalline or amorphous form.

17. he method of claim 13, wherein the direct-acting alpha 2 adrenegic agonist is selected from a group consisting of Apraclonidine, Brimonidine, Clonidine, Detomidine, Dexmedetomidine, Guanabenz, Guanfacine, Lofexidine, Medetomidine, Romifidine, Tizanidine, Tolonidine, Xylazine and Fadolmidine.

18. The method claim 13, wherein the direct-acting alpha 2 adrenegic agonist is clonidine or its pharmaceutically acceptable salt.

19. The method of claim 13, wherein the opioid antagonist is a quantity of naltrexone in combination with second pharmaceutical agent selected from a group consisting of a non steroidal anti inflammatory drug (NSAID) of all chemical groups, including COX-2 selective inhibitor(coxibs), steroidal anti inflammatory drugs, Tricyclic antidepressants (TCAs), Selective serotonin reuptake inhibitors (SSRIs), Serotonin-norepinephrine reuptake inhibitors (SNRIs), Anticonvulsants, muscle relaxant, drug with NMDA antagonist properties, Tetrahydrocannabinol derivatives, an antitussive, an expectorant, a decongestant, or an antihistamine.

20. The method of claim 13, further including the step of administering a dosage during the daytime, and then a second dosage proximate to bedtime.
